(19) United States
(12) Patent Application Publication
(10) Pub. No.: US 2024/0168207 A1
(43) Pub. Date: May 23, 2024

(54) FABRICATION OF SLANTED GRATING MASTER AND WORKING STAMP USING GRAYSCALE LITHOGRAPHY AND PLASMA ETCHING

(71) Applicant: GOOGLE LLC, Mountain View, CA (US)

(72) Inventors: Lu Tian, Palo Alto, CA (US); Wei Jin, Saratoga, CA (US); Thomas Mercier, Weston, FL (US)

(21) Appl. No.: 18/489,397

(22) Filed: Oct. 18, 2023

Related U.S. Application Data
(60) Provisional application No. 63/427,720, filed on Nov. 23, 2022.

(51) Int. Cl. G02B 5/18; F21V 8/00; G02B 27/01

(52) U.S. Cl. CPC G02B 5/1857; G02B 6/0065; G02B 27/0172; G02B 2027/0178

(57) ABSTRACT

A method for fabricating a working stamp for forming slanted surface gratings in a waveguide workpiece includes providing a master workpiece comprising a substrate and performing a sequence of photoresist deposition processes, grayscale lithography processes, and etching processes on the master workpiece so as to form an imprint replication master having a pattern of slanted gratings in a working surface of its substrate, the slanted gratings having sidewalls that are not substantially orthogonal to a working surface of the substrate. The method also includes conforming a soft stamp material layer to the working surface of the imprint replication master so that the soft stamp material layer has a pattern of slanted protrusions corresponding to the pattern of slanted gratings, and removing the imprint replication master from the soft stamp material and curing the soft stamp material layer surrounding the pattern of slanted protrusions to form the working stamp.

### CROSS-REFERENCE TO RELATED APPLICATIONS

The present application claims priority to U.S. Provisional Application No. 63/417,720, entitled “FABRICATION OF SLANTED GRATING MASTER AND WORKING STAMP USING GRAYSCALE LITHOGRAPHY AND PLASMA ETCHING” and filed on Oct. 20, 2022, the entirety of which is incorporated by reference herein.

### BACKGROUND

Head-mounted devices (HMDs), heads-up displays (HUDs) and other near-eye display systems often employ waveguides that utilize surface gratings or holographic gratings for various light manipulation purposes, such as the incoupling of display light into the waveguide or the outcoupling of display light from the waveguide toward the direction of a user's eye. Slanted surface gratings typically have a much higher diffraction efficiency compared to binary surface gratings and blazed surface gratings, and thus are well suited for augmented reality (AR) and virtual reality (VR) waveguide applications. However, compared to binary surface gratings and blazed surface gratings and their orthogonal, or “vertical”, sidewalls, slanted gratings are relatively difficult to fabricate using conventional fabrication techniques due to their non-orthogonal relationship to the workpiece surface during the fabrication process.

### BRIEF DESCRIPTION OF THE DRAWINGS

The present disclosure may be better understood, and its numerous features and advantages made apparent to those skilled in the art by referencing the accompanying drawings. The use of the same reference symbols in different drawings indicates similar or identical items.

FIG. 1 illustrates an initial stage of a method for fabrication of a working stamp having slanted gratings in accordance with implementations.

FIG. 2 illustrates an intermediate stage of the method of FIG. 1 in accordance with implementations.

FIG. 3 illustrates a final stage of the method of FIG. 1 in accordance with implementations.

FIG. 4 illustrates a method for fabrication of slanted gratings in a waveguide workpiece using a working stamp in accordance with implementations.

FIG. 5 illustrates a perspective rear view of AR glasses having at least one waveguide with slanted gratings fabricated using the method of FIGS. 1-4 in accordance with implementations.

### DETAILED DESCRIPTION

A common approach to fabrication of a waveguide with surface gratings relies on the use of a working stamp that has the negative, or inverse, pattern of the intended pattern of the surface gratings. The working stamp is pressed into the appropriate location on the surface of a waveguide workpiece to form the corresponding surface grating pattern at that surface of the waveguide workpiece. After withdrawing the working stamp from the waveguide workpiece, a curing process then may be applied to at least the area in which the slanted gratings were formed so as to cure and harden the slanted gratings. To form the working stamp itself, an imprint replication master is fabricated, with the imprint replication master itself having the same pattern as the gratings to be formed in the waveguide workpiece, which is the negative, or inverse, pattern of the working stamp. The imprint replication master is pressed into a workpiece composed of suitable working stamp material so as to form the negative/inverse grating pattern in the workpiece, and then the workpiece is cured, resulting in the working stamp.

For gratings with vertical sidewalls (that is, sidewalls that are approximately orthogonal to the waveguide workpiece surface in which the gratings are formed), such as binary gratings and blaze gratings, forming the imprint replication master is a relatively straightforward process because the sidewalls of the imprint pattern are vertical, and thus well suited to conventional photolithography processes. However, slanted gratings (that is, gratings with sidewalls that are not approximately orthogonal to the waveguide workpiece surface in which the gratings are formed) provide improved diffraction efficiency in an indicated direction compared to vertical gratings by the nature of their non-vertical slant. However, this non-vertical slant results in non-vertical sidewalls. Fabricating an imprint replication master that incorporates these non-vertical slanted gratings introduces additional challenges in angle control, parallelism of positive and negative sidewall angles, lateral etching control, surface roughness control, large area pattern uniformity, and the like. Conventional fabrication approaches in which the workpiece surface is oriented non-orthogonally to a plasma dry etching process often do not adequately overcome these challenges, resulting in a sub-optimal imprint replication master or additional remedial fabrication processes to address the issues noted above.

To overcome the challenges inherent to fabrication of an imprint replication master and resulting working stamp for stamping slanted gratings into a waveguide workpiece, the following describes implementations of a fabrication process in which one or more slanted grating structures are formed in a waveguide workpiece using a working stamp fabricated by performing a sequence of photoresist deposition processes, grayscale lithography processes, and etching processes on a master workpiece. As such, the grating structure formed in this way benefits from the use of established vertical plasma etching processes that can be performed using any of a variety of typical RIE (reactive ion etching) etching systems or similar etching systems. Furthermore, the slanted angles of the grating structures can be well defined by the grayscale lithography process, which mitigates the aforementioned challenges of angle control, parallelism, lateral etching control, surface roughness control, and the like.

Used herein are various position-based or orientation-based terms, such as “vertical”, “horizontal”, “top”, “bottom”, and the like. It will be appreciated that these terms are used merely with reference to the orientation of the view of the corresponding figure, and are not intended to specifically describe a particular orientation with respect to a gravitational reference unless otherwise noted.

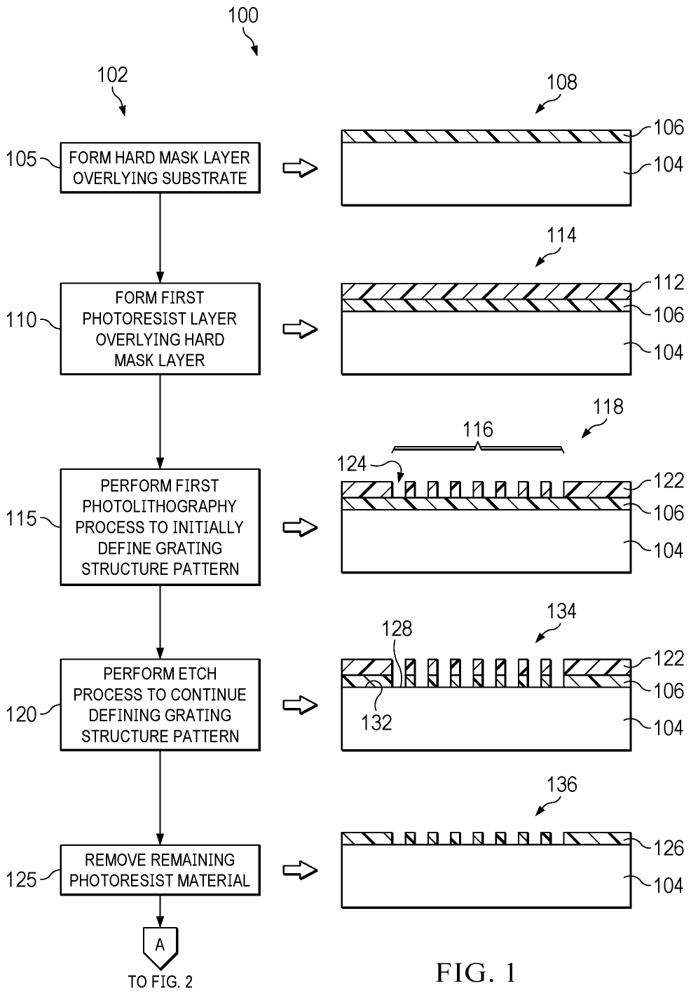

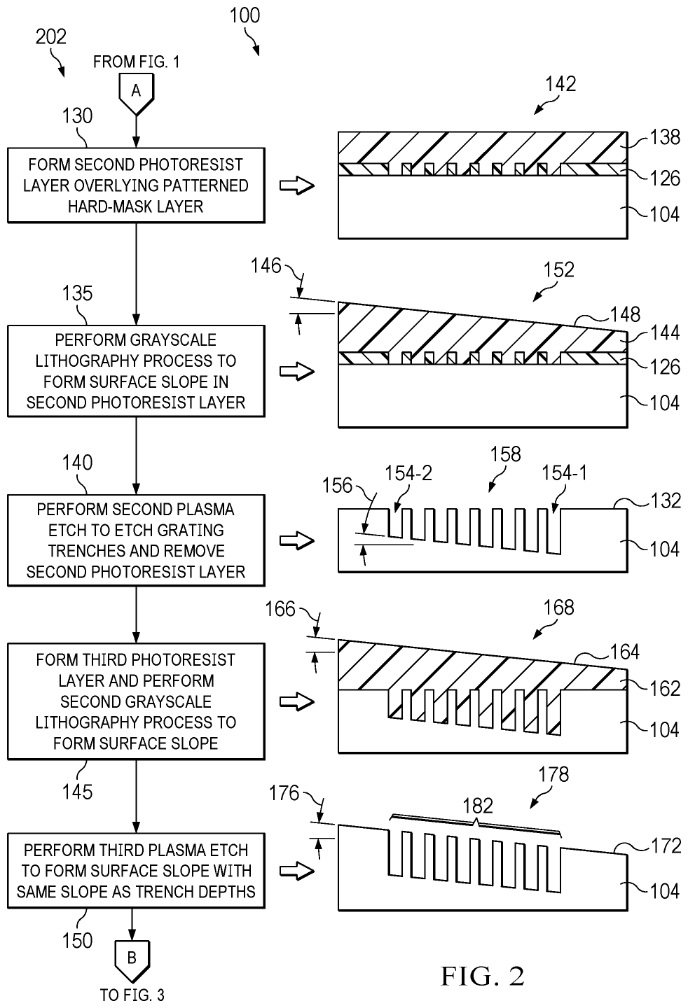

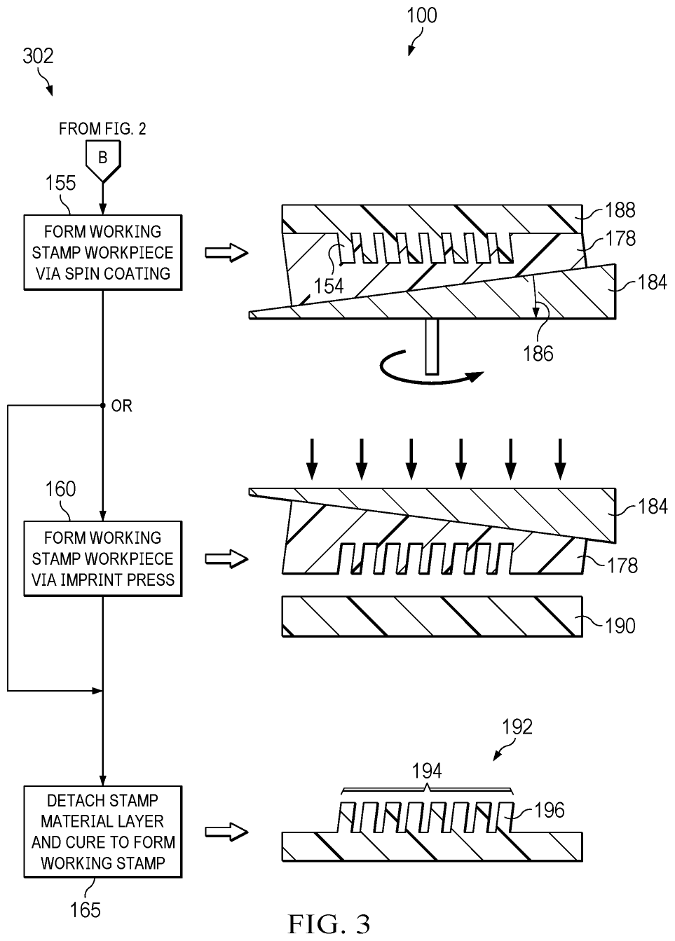

FIGS. 1-3 illustrate an initial stage 102 (FIG. 1), an intermediate stage 202 (FIG. 2) and a final stage 302 (FIG. 3) of a method 100 for fabricating an imprint replication master for fabrication of slanted gratings in a waveguide workpiece in accordance with implementations. In the depicted example, the initial stage 102 begins with the provision of a suitable substrate 104 for fabrication of an imprint replication master, such as a quartz or silicon substrate, followed by formation of a hard-mask layer 106 overlying the substrate 104, resulting in master workpiece 108. The hard-mask layer 106 may be composed of a metal, a dielectric, or a combination thereof and formed using, for example, a photoresist deposition process. Examples of the material of the hard-mask layer 106 include, for example, silicon dioxide, silicon carbide, amorphous carbon, titanium nitride, tantalum nitride, and the like. At block 110, a photoresist layer 112 is formed overlying the hard-mask layer 106, resulting in master workpiece 114. In implementations, the photoresist layer 112 is formed using a nanopatterning process in which the photoresist layer 112 has nanostructures (that is, structures with one or more dimensions below 100 micrometers) formed at the top surface of the photoresist layer 112.

At block 115, a first photolithography process is performed on the master workpiece 114 to initiate the process of defining an initial grating structure pattern 116, resulting in a master workpiece 118. In particular, the photolithography process is configured to remove portions of the photoresist layer 112 in areas to expose the hard-mask layer 106, resulting in a pattern of openings in a patterned photoresist layer 122, with the exposed areas (e.g., exposed area 124) being positioned and dimensioned to match the width, length, and positions of the tops of slanted gratings in the initial slanted grating pattern 116 to be formed. At block 120, the grating structure pattern continues to be defined by performing a vertical plasma etch process to remove the material of the hard-mask layer 106 exposed through the patterned openings in the patterned photoresist layer 122, thereby resulting in a pattern of openings in a patterned hard-mask layer 126 that exposes areas (e.g., area 128) of a top surface 132 of the substrate 104, resulting in master workpiece 134. At block 125, the remaining photoresist material of the patterned photoresist layer 122 is removed from the master workpiece 134, resulting in a master workpiece 136 having the substrate 104 with the overlying patterned hard-mask layer 126.

Referring now to FIG. 2, the intermediate stage 202 of the method 100 begins. Following the process of block 125 from FIG. 1 above, at block 130 a photoresist deposition process is performed to form a second photoresist layer 138 overlying the patterned hard-mask layer 126 and the underlying exposed surfaces of the substrate 104 of the master workpiece 136, resulting in master workpiece 142. In implementations, the photoresist layer 138 is a low-contrast photoresist layer that has a linear thickness response to exposure dosage after development, which facilitates formation of a slope in the photoresist layer 138, as described next. At block 135, a grayscale lithography technique is performed on the master workpiece 142 with the appropriate exposure conditions so as to define a resist surface slope in the low-contrast photoresist layer 138, resulting in a sloped photoresist layer 144 with a resulting slope 146 in the top surface 148 of the slanted photoresist layer 144 matching the “slant” of the intended slanted gratings. That is, the top surface 148 of the slanted photoresist layer 144 is non-parallel (that is, at a non-zero angle, or slant, 146) to the top surface 132 of the substrate 104, with this slant/slope 146 of the top surface 148 being substantially equal to the intended angle of the sidewalls of the slanted gratings to be formed using the imprint replication master to be formed through method 100. As understood in the art, a grayscale lithography technique, such as the one performed at block 135, provides light with spatial variation of exposure dosage (that is, grayscale variation) on the low-contrast photoresist material of the photoresist layer 138, and the photoresist polymer of the photoresist layer 138 is exposed and developed in developer chemical solution, which due to the spatial variation of the exposure dosage, results in the imparted slope 146 in the top surface 148 of the slanted photoresist layer 144. As described below, after developing, the developed partial of photoresist can be removed and the un-developed portion remains on the substrate.

At block 140, a second vertical plasma etch process is performed on the master workpiece 152 resulting from block 135 so as to etch grating trenches 154 (e.g., grating trenches 154-1 and 154-2) into the top surface 132 of the substrate 104 and the slanted photoresist layer 144 is removed. As will be understood, the grating trenches 154 are formed in the areas where the top surface 132 of the substrate 104 is exposed through the pattern of openings etched into the patterned hard-mask layer 126 such that the width, length, and position of each grating trench 154 corresponds to the width, length, and position of a corresponding opening formed in the patterned hard-mask layer 126 during block 125. Moreover, as also will be understood, the depth of each trench is inversely proportional to the thickness of the slanted photoresist layer 144 overlying the position of the grating trench 154. Thus, the grating trench 154-2 formed where the slanted photoresist layer 144 is thickest will be the shallowest grating trench 154, whereas the grating trench 154-1 formed where the slanted photoresist layer 144 is thinnest will be the deepest grating trench. The depth “slope” 156 between the grating trenches 154 thus matches the surface slope 146 of the slanted photoresist layer 144, and thus corresponds to the slant angle intended for the slanted gratings to be formed.

At block 145, the a photoresist process is performed to form a third (low-contrast) photoresist layer on top surface of the resulting master workpiece 158 and a second grayscale lithography process is performed using the same exposure conditions as at block 135 so as to form a slanted photoresist layer 162 with a top surface 164 that has a slope 166 (that is, a non-zero angle relative to the top surface 132 of the substrate 104) that substantially matches the surface slope 146 of the slanted photoresist layer 144 formed at block 135. As such, the surface slope 166 of the slanted photoresist layer 162 in the resulting master workpiece 168 substantially matches the slope 156 of the underlying grating trenches formed in the substrate 104.

At block 150, a third vertical plasma etching process is performed on the master workpiece 168 to define a resulting top surface 172 of the substrate 104 as having a slope 176 that is substantially the same as the slope 156 of the depths, or bottoms, of the grating trenches 154. Further, the overlying photoresist layer 162 is removed, a wafer cleaning process is performed, and then an anti-stick surface treatment is applied to the top surface 172 of the substrate 104 (including the sidewalls and bottom surfaces of the grating trenches 154). The result of the process of blocks 105-150 is an imprint master 178 with a slanted grating pattern 182 representing the slanted gratings intended for fabrication in a corresponding waveguide workpiece.

Referring now to FIG. 3, the final stage 302 of the method 100 begins. In this stage, the imprint master 178 resulting from the process of blocks 105-150 is used to form a working stamp in accordance with implementations. In some implementations, at block 155, the imprint master 178 is mounted to an angle compensation chuck 184 via, e.g., vacuum mounting, where the angle compensation chuck 184 has a slope 186 complementary to the slope 176 of the top surface 172 of the imprint master 178 such that when the bottom surface of the imprint master 178 is mounted to the angle compensation chuck 184, the top surface 172 of the imprint master 178 provides a “horizontal” working surface for a spin coating process that is then performed to spin coat the imprint master with an overlying soft stamp material layer 188 (e.g., uncured polymer) that forms a conformal layer over the top surface 172 of the imprint master 178 and also extends to the bottom of each grating trench 154 in the imprint master 178.

Alternatively, instead of using a spin coating process, at block 160 the imprint master 178 with the angle compensation chuck 184 attached is inverted and the imprint master 178 is downward pressed into a soft stamp material layer 190 (e.g., uncured polymer), thereby causing the soft stamp material to form a conformal stamp material layer that conforms to the surface of the imprint master 178, including the grating trenches 154. In either approach, after the soft stamp material layer 188 or 190 is formed to conform to the imprint master 178, at block 165 the stamp material layer 188 or 190 is detached from the imprint master 178 and then cured to harden the material, thereby forming a working stamp 192 having a pattern 194 of slanted protrusions 196 in the negative/inverse slanted grating pattern 198 to be employed in forming slanted gratings in a waveguide workpiece. In implementations, the soft stamp material layer 188 or 190 is detached from the imprint master 178 and then cured, and in other embodiments the soft stamp material layer 188/190 is partially cured while in contact with the imprint master 178 so as to set the imprinted pattern, and then fully cured once the partially-cured layer is removed from the imprint master 178.

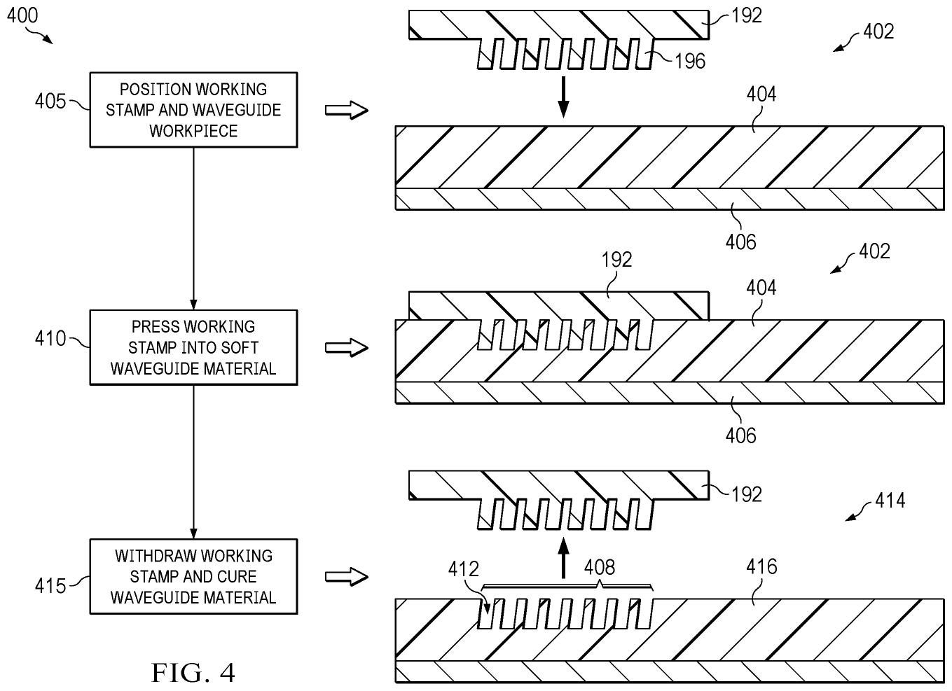

FIG. 4 illustrates an example fabrication method 400 for forming slanted gratings in a waveguide workpiece using the working stamp 192 formed according to the method 100 of FIGS. 1-3 in accordance with implementations. As shown, at block 405 a waveguide workpiece 402 composed of a soft waveguide material layer 404 (e.g., uncured polymer) is formed on a stiff support carrier 406, and then the working stamp 192 is oriented so that the slanted protrusions 196 (representing the negative, or inverse, slanted gratings pattern to be formed in the waveguide workpiece 402) face the soft waveguide material layer 404 and are positioned overlying the region in which the slanted gratings are to be formed. At block 410, the working stamp 192 is coated with an anti-stick material and then pressed into the facing surface of the soft waveguide material layer 404, causing the soft waveguide material layer 404 to conform to the working stamp 192, and in particular, to form slanted gratings in the soft waveguide material layer 404 due to conformation to the slanted protrusions 196. At block 415, the working stamp 192 is then removed from the soft waveguide material layer 404, resulting in a slanted gratings pattern 408 of a set of slanted gratings 412 being formed in the soft waveguide material layer 404. A curing process is performed for at least the impacted region of the soft waveguide material layer 404 to retain the slanted gratings pattern 408. In implementations, the soft waveguide material layer 404 can be partially cured while the working stamp 192 is in place so as to partially harden the waveguide material that forms the slanted gratings 412 and then the region is subjected to a second cure process to fully cure the region after the working stamp 192 is withdrawn. In other embodiments, the working stamp 192 is withdrawn first and then a full cure process is performed. The result is a waveguide workpiece 414 having the pattern 408 of slanted gratings 412 formed at the top surface 416.

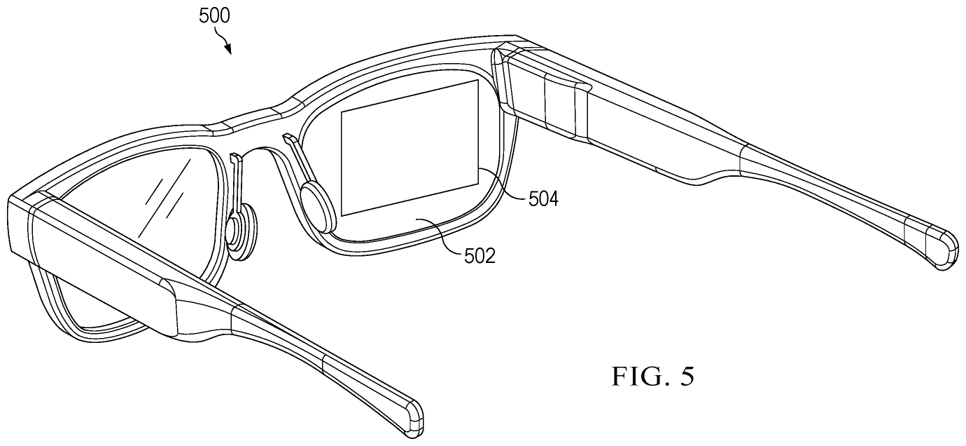

Thereafter, the waveguide workpiece 414 may be subjected to one or more additional fabrication processes (including removal from the carrier 406), resulting in a waveguide having slanted gratings for use in an optical system. FIG. 5 illustrates one such optical system in the form of a set of AR glasses 500 implementing a waveguide having slanted surface gratings formed via use of a working stamp formed via an imprint master in accordance with FIGS. 1-4 above. As shown, the AR glasses 500 include a set of lenses, including a lens 502 incorporating a waveguide 504 having surface gratings formed therein in accordance with the processes described above, such as for an incoupler, an outcoupler, or some other optical component of the waveguide.

In implementations, certain aspects of the techniques described above may be implemented by one or more processors of a processing system executing software. The software comprises one or more sets of executable instructions stored or otherwise tangibly embodied on a non-transitory computer readable storage medium. The software can include the instructions and certain data that, when executed by the one or more processors, manipulate the one or more processors to perform one or more aspects of the techniques described above. The non-transitory computer readable storage medium can include, for example, a magnetic or optical disk storage device, solid state storage devices such as Flash memory, a cache, random access memory (RAM) or other non-volatile memory device or devices, and the like. The executable instructions stored on the non-transitory computer readable storage medium may be in source code, assembly language code, object code, or other instruction format that is interpreted or otherwise executable by one or more processors.

A computer readable storage medium may include any storage medium, or combination of storage media, accessible by a computer system during use to provide instructions and/or data to the computer system. Such storage media can include, but is not limited to, optical media (e.g., compact disc (CD), digital versatile disc (DVD), Blu-Ray disc), magnetic media (e.g., floppy disc, magnetic tape, or magnetic hard drive), volatile memory (e.g., random access memory (RAM) or cache), non-volatile memory (e.g., read-only memory (ROM) or Flash memory), or microelectromechanical systems (MEMS)-based storage media. The computer readable storage medium may be embedded in the computing system (e.g., system RAM or ROM), fixedly attached to the computing system (e.g., a magnetic hard drive), removably attached to the computing system (e.g., an optical disc or Universal Serial Bus (USB)-based Flash memory), or coupled to the computer system via a wired or wireless network (e.g., network accessible storage (NAS)).

Note that not all of the activities or elements described above in the general description are required, that a portion of a specific activity or device may not be required, and that one or more further activities may be performed, or elements included, in addition to those described. Still further, the order in which activities are listed are not necessarily the order in which they are performed. Also, the concepts have been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims. Moreover, the particular embodiments disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular embodiments disclosed above may be altered or modified and all such variations are considered within the scope of the disclosed subject matter. Accordingly, the protection sought herein is as set forth in the claims below.

What is claimed is:

1. A method for fabricating a working stamp for forming slanted surface gratings in a waveguide workpiece, comprising:
providing a master workpiece comprising a substrate; and
performing a sequence of photoresist deposition processes, grayscale lithography processes, and etching processes on the master workpiece so as to form an imprint replication master having a pattern of slanted gratings in a working surface of the substrate, the slanted gratings having sidewalls that are not substantially orthogonal to a working surface of the substrate;
conforming a soft stamp material layer to the working surface of the substrate of the imprint replication master so that the soft stamp material layer has a pattern of slanted protrusions corresponding to the pattern of slanted gratings; and
removing the imprint replication master from the soft stamp material and curing at least an area of the soft stamp material layer surrounding the pattern of slanted protrusions to form the working stamp.

2. The method of claim 1, wherein the sequence of photoresist deposition processes, grayscale lithography processes, and etching processes comprises:
forming a hard-mask layer overlying the working surface of the substrate;
forming a first photoresist layer overlying the hard-mask layer;
performing a first photolithography process to form a first pattern of openings in the first photoresist layer, the first pattern of openings corresponding to a pattern of surface gratings;
performing a first etch process to form a second pattern of openings in the hard-mask layer to generate a patterned hard-mask layer, the second pattern of openings corresponding to the first pattern of openings; and
removing the first photoresist layer.

3. The method of claim 2, wherein the first photoresist layer is a nano-pattern photoresist layer.

4. The method of claim 2, wherein the sequence of photoresist deposition processes, grayscale lithography processes, and etching processes comprises:
forming a second photoresist layer overlying the patterned hard-mask layer;
performing a first grayscale lithography process to form a slope, relative to the working surface, in a surface of the second photoresist layer that is opposite the working surface; and
performing a second etch process to etch a pattern of grating trenches at the working surface of the substrate, wherein depths of the grating trenches have a substantially same slope as a slope in the surface of the second photoresist layer.

5. The method of claim 4, wherein the second photoresist layer is a low-contrast photoresist layer.

6. The method of claim 4, wherein the first and second etch processes are vertical plasma etch processes.

7. The method of claim 4, wherein the sequence of photoresist deposition processes, grayscale lithography processes, and etching processes comprises:
forming a third photoresist layer overlying the working surface;
performing a second grayscale lithography process to form a slope, relative to the working surface, in a surface of the third photoresist layer that is opposite the working surface; and
performing a third etch process to form a slope in the working surface that is substantially the same as the slope in the depths of the grating trenches so as to generate the imprint replication master.

8. The method of claim 7, wherein conforming the soft stamp material layer to the working surface comprises:
performing a spin-coating process to coat the working surface of the substrate of the imprint replication master with a conformal layer of soft stamp material; and
curing the conformal layer of soft stamp material to form the working stamp.

9. The method of claim 7, wherein conforming the soft stamp material layer to the working surface comprises:
pressing a working surface of a substrate of the imprint replication master into a layer of soft stamp material to form a conformal layer of soft stamp material; and
curing the conformal layer of soft stamp material to form the working stamp.

10. The method of claim 1, wherein the sequence of photoresist deposition processes, grayscale lithography processes, and etching processes comprises:
forming a photoresist layer overlying a patterned hard-mask layer that overlies the working surface of the substrate, the patterned hard-mask layer having a pattern of openings that expose corresponding areas of the working surface of the substrate;
performing a grayscale lithography process to form a slope in a first surface of the photoresist layer relative to the working surface of the substrate; and
performing an etch process to etch a pattern of grating trenches at the working surface of the substrate, wherein depths of the grating trenches have a substantially same slope as a slope in the first surface of the photoresist layer.

11. The method of claim 1, wherein conforming the soft stamp material layer to the working surface comprises:
performing a spin-coating process to coat a working surface of a substrate of the imprint replication master with a conformal layer of soft stamp material; and
curing the conformal layer of soft stamp material to form the working stamp.

12. The method of claim 1, wherein conforming the soft stamp material layer to the working surface comprises:
pressing a working surface of a substrate of the imprint replication master into a layer of soft stamp material to form a conformal layer of soft stamp material; and
curing the conformal layer of soft stamp material to form the working stamp.

13. A working stamp for forming slanted gratings in a waveguide workpiece fabricated in accordance with the method of claim 1.

14. A waveguide workpiece having slanted gratings formed therein using the working stamp of claim 1.

15. A method for fabricating slanted surface gratings in a waveguide workpiece, comprising:
pressing a working stamp having a pattern of slanted protrusions extending from a first surface into an opposing second surface of a layer of soft waveguide material of the waveguide workpiece so that the slanted protrusions extend into the layer of soft waveguide material;
withdrawing the working stamp from the layer of soft waveguide material so that a pattern of slanted surface gratings are formed in the layer of soft waveguide material at the second surface; and
curing at least an area of the layer of soft waveguide material surrounding the pattern of slanted gratings.

16. The method of claim 15, further comprising:
forming the working stamp from an imprint replication master having a plurality of slanted protrusions by:
performing a spin-coating process to coat a working surface of a substrate of the imprint replication master with a conformal layer of soft stamp material; and
curing the conformal layer of soft stamp material to form the working stamp.

17. The method of claim 15, further comprising:
forming the working stamp from an imprint replication master having a plurality of slanted protrusions by:
pressing a working surface of a substrate of the imprint replication master into a layer of soft stamp material to form a conformal layer of soft stamp material; and
curing the conformal layer of soft stamp material to form the working stamp.

18. The method of claim 15, further comprising:
forming an imprint replication master using a grayscale lithography process to introduce a slope into a surface of a photoresist layer of a master workpiece, the slope based on a slant of the slanted gratings; and
forming the working stamp from the imprint replication master.

19. A waveguide workpiece having slanted gratings formed therein using the working stamp of claim 18.

20. A waveguide workpiece having slanted gratings formed therein using the working stamp of claim 15.
